(19) United States
(12) Patent Application Publication
(10) Pub. No.: US 2009/0216167 A1
(43) Pub. Date: Aug. 27, 2009

(54) ANKLE SPRAIN REDUCTION SYSTEM

(76) Inventor: Nathaniel Harris, Lewisburg, WV (US)

Correspondence Address: CONNOLLY BOVE LODGE & HUTZ, LLP, P O BOX 2207, WILMINGTON, DE, 19899, US

(21) Appl. No.: 12/036,532

(22) Filed: Feb. 25, 2008

(51) Int. Cl. A61F 5/00

(52) U.S. Cl. 602/27; 36/89

(57) ABSTRACT

An ankle sprain reduction system includes a shoe having a brace incorporated in the shoe. The brace has a base plate with medial and lateral sides extending upwardly from a lower base portion. Medial and lateral support plates are pivotally mounted to each of the sides at a location below the malleoli. The lower base portion is of a length that extends from the heel to just proximal to the metatarsalphalangeal joints of the foot.

### BACKGROUND OF INVENTION

The present invention relates to reducing the severity of and preventing ankle sprain occurrences. Such ankle sprains are often encountered by individuals participating in athletics, although, such sprains can be encountered under other circumstances. Two types of sprains that occur are hyperextensions and inversion strains. These types of sprains might result where, for example, the toes are bent too far causing hyperextension. Other causes might result from stepping on an object which would cause a twisting of the ankle. It would be desirable if some form of brace or system could be provided to reduce the occurrence and severity of ankle sprains.

Attempts have been made to address such ankle sprains in various manners. One general approach that has been used is to provide an ankle brace, such as one that might be customized for the particular wearer. Such brace would be separate and distinct from the footwear and although it is intended to be customized for a particular wearer, it does not provide the desired degree of reducing the occurrence and severity of ankle sprains.

### SUMMARY OF INVENTION

An object of this invention is to provide an ankle sprain reduction system which is incorporated in the actual footwear or shoe.

A further object of this invention is to provide such an ankle sprain reduction system which is applied by the user simply placing the foot into the shoe in a conventional manner and wherein protection is always present as long as the shoe is laced snuggly.

In accordance with this invention the ankle sprain reduction system includes brace having a base plate and medial and lateral support plates. The base plate is incorporated into and becomes part of the footwear or shoe by being located in the sole of the shoe and preferably in a recess in the sole. This creates a smooth sole surface. The base plate has medial and lateral sides extending upwardly from its lower base portion and extending below the respective malleolus terminating at the back of the shoe. The lateral and medial support plates are pivotally mounted to the lateral and medial side extensions of the base plate. The base plate is of a length to extend from about the heel to just proximal to the metatarsalphalangeal joints. In this manner, there is a maximum area of protection for the foot without interfering with toe movement.

To enhance comfort padding is provided on the surfaces of the base plate and lateral and medial support plates which contact the user's foot. Such padding could be an insole which extends over both the sole and the lower base portion of the base plate and could be padding lining the inner surfaces of the base plates sides and of the support plates. This padding could be the same as that already found in typical athletic shoes.

The lateral and medial support plates would be outwardly bulged to accommodate the malleolus. Each support plate may be pivotally mounted to its respective base plate side by a pin which could be integral with the respective base plate side. The pin could be of a size and/or selected from a material that is capable of breaking if excessive force is encountered which when transferred to the lower leg exceeds the bone strength and would result in fracture. The breakaway pin would break before the fracture threshold force is transferred to the tibia.

### THE DRAWINGS

FIG. 1 and FIG. 1A are side views of a human foot in a condition which would be subject to hyperextension;

FIG. 1B is a front view of a human foot in a condition which would be subject to inversion strains;

FIG. 2 is a side view, partly in section, from the lateral side of an ankle sprain reduction system in accordance with this invention;

FIG. 3 is a front view of the brace portion of the ankle sprain reduction system shown in FIG. 2;

FIG. 4 is a top plan view of the brace portion shown in FIG. 3;

FIG. 5 is a side view, partly in section, from the medial side of the ankle sprain reduction system shown in FIGS. 2-4;

FIG. 6 is a cross-sectional view taken through FIG. 2 along the lines 6-6, but with the shoe omitted;

FIG. 7 is a cross-sectional view taken through FIG. 2 along the lines 7-7, but with the shoe omitted;

FIG. 8 is an enlarged view in section showing the pivotal mounting of the lateral components of the ankle sprain reduction system of FIGS. 2-7;

FIG. 9 is a top plan view showing the sole portion of a shoe which is part of the ankle sprain reduction system of this invention;

FIG. 10 is a top plan view of the ankle sprain reduction system of FIGS. 2-9;

FIG. 11 is a top plan view of an insole that could be used in the ankle sprain reduction system of this invention; and

FIG. 12 is a top plan view showing a shoe incorporating the ankle sprain reduction system of this invention.

### DETAILED DESCRIPTION

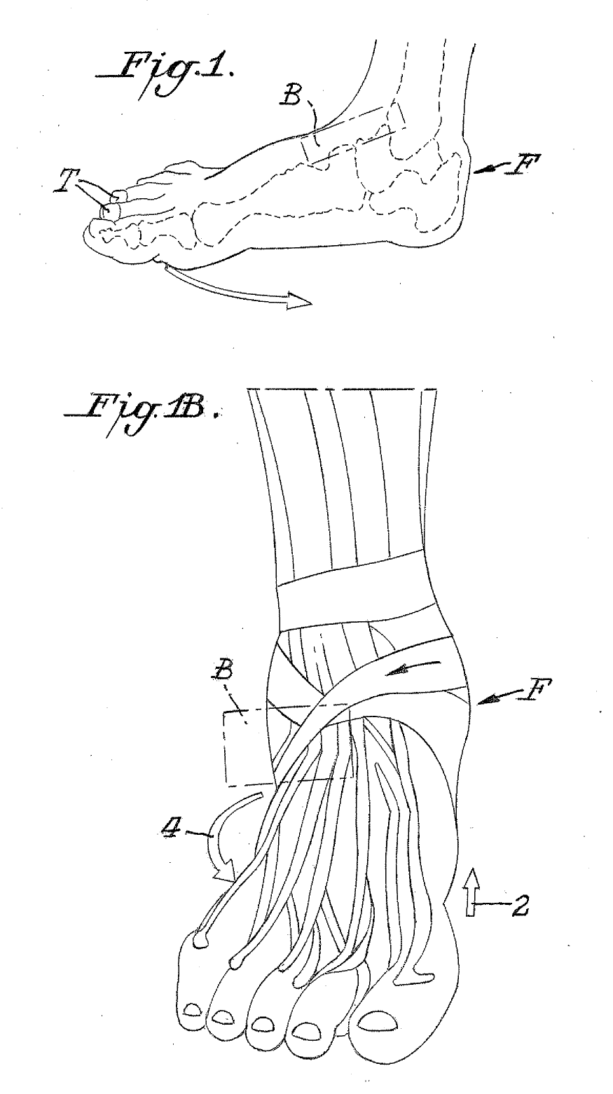

FIGS. 1 and 1A illustrate a human foot F which is in a condition that may be subject to hyperextension. If the toes T are pointed downwardly against the ground G, as particularly shown in FIG. 1A, the damaging forces (illustrated by the arrow 1) into the heel, strain the anterior ligaments, particularly in the area of the box B shown in FIG. 1. In order to avoid an ankle sprain resulting from hyperextension it would be desirable to provide some structure for preventing further backwards motion.

FIG. 1B shows the condition of a foot F which would be subject to inversion strains. Such inversion strains generally result from an upward force on the medial side of the foot from beneath as indicated by the arrow 2. This causes the foot to rotate as indicated by the arrow 4. Such rotation could result in ligament strain in the area indicated by the box B.

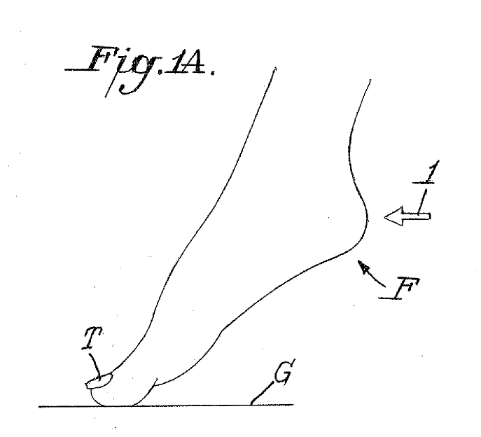

FIGS. 2-12 illustrate an ankle sprain reduction system 10 in accordance with this invention. System 10, in general, includes two major components. One of these components is an article of footwear which will be referred to as shoe 12. The other component is a brace 14 which (in the preferred practice of this invention) is incorporated into the shoe 12 so as to become a permanent part of the shoe as later described. The shoe 12, itself, could be any suitable type of footwear adaptable to receive the brace. Examples of such footwear include sneakers, tennis shoes, high-tops, boots, etc. in addition to various types of conventional shoes. Accordingly, the term “shoe” is used in its broadest sense to include any such types of footwear. While the invention will be described with respect to a single shoe, the invention is preferably practiced with a pair of shoes, namely, one for the right foot and the other for the left foot. In addition, because one advantage of the invention is that a brace is made to fit a particular size shoe, the invention could be practiced where there are a plurality of sets of shoes with each set corresponding to a different size so that a user can select the proper size in the same manner that a user would ordinarily select a proper size shoe which does not include a brace.

The shoe 12 would have conventional components, some of which would be modified to be adapted to the brace. Such components include a sole 16 and an insole 18 which provides cushioning over the sole. The shoe would also, preferably, include an upper which has an ankle portion 20. In addition, as illustrated in FIG. 12 the shoe is preferably one which could be tightened to snuggly fit on the wearer once the foot is inserted. Thus, FIG. 12 illustrates eyelets or holes 22 for receiving a lace in a conventional manner. Other forms of adjustable snug fastening structure could also be used instead of laces.

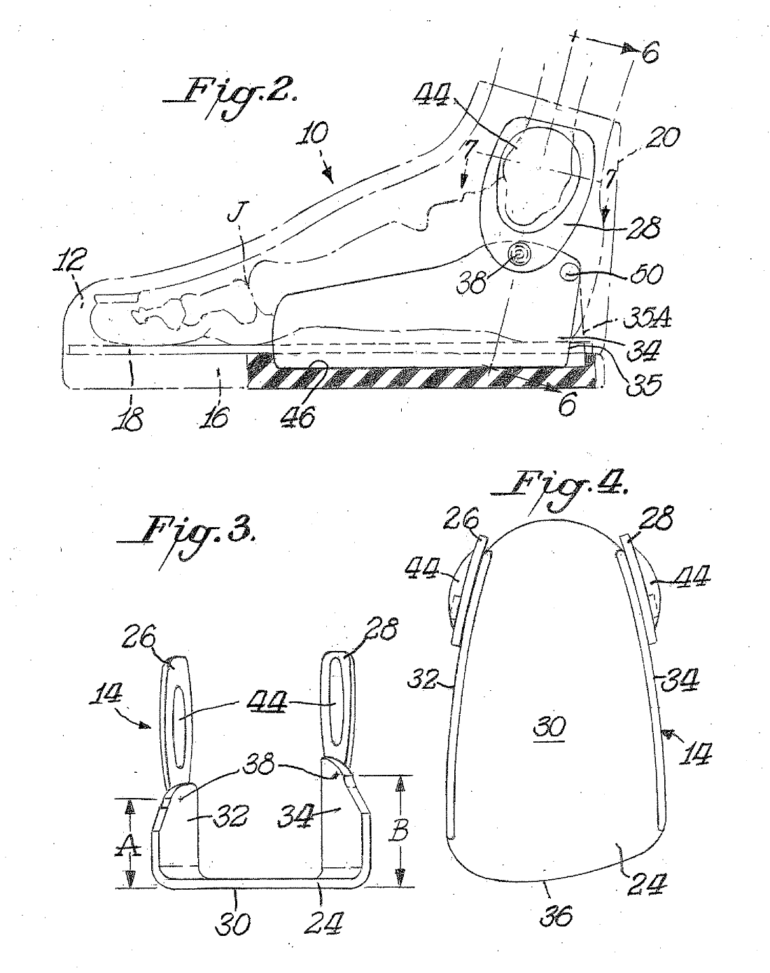

As illustrated in FIGS. 3-4 the brace 14 essentially comprises three main components. One of these components is a base plate 24. A second component is a medial support plate 26. The third component is a lateral support plate 28. The base plate 24 and the support plates 26 and 28 are made of a rigid material.

As shown in FIG. 3 base plate 24 includes a lower base portion 30 and further includes a medial side 32 extending upwardly from the base portion 30 to a distance just below the medial malleolus as shown in FIG. 5, and extending longitudinally to the end of the sole at the heel. Medial side 32 is pivotally connected to medial support plate 26 so that the medial support plate extends upwardly from the medial side 32 at a location just vertically below the medial malleolus to a distance vertically above the medial malleolus. The extent that medial support plate is vertically above the medial malleolus is one wherein the support plate would be confined within the shoe. What is important is that the medial support plate covers and protects the medial malleolus and the distal fibula.

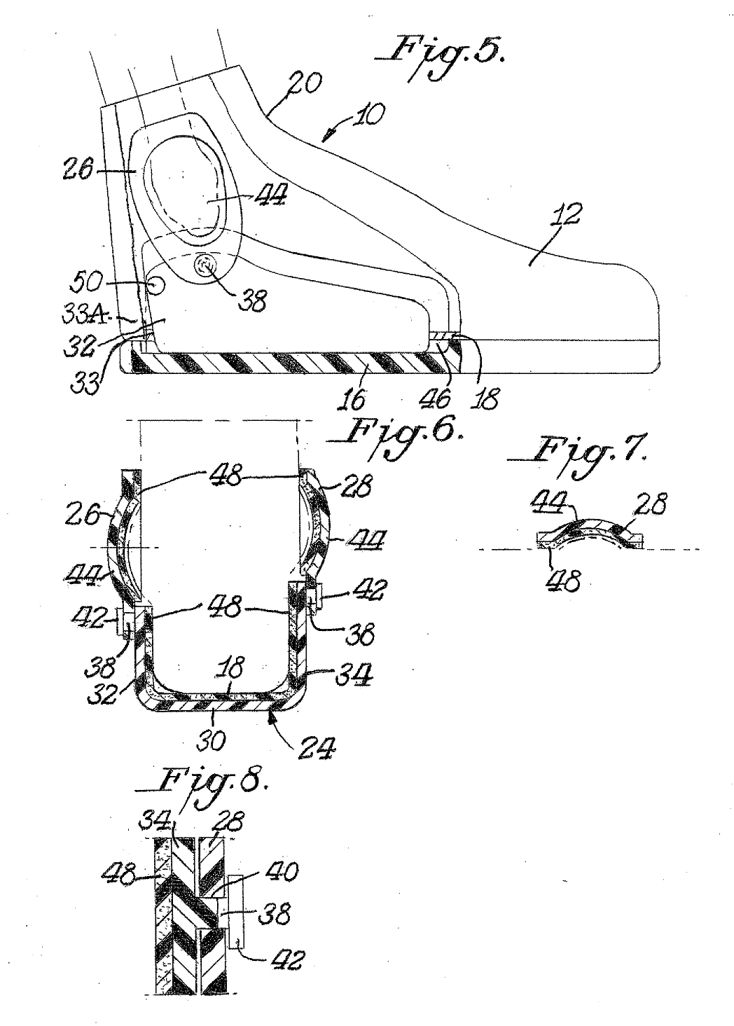

As also shown in FIGS. 2-3 a lateral side 34 extends upwardly from the base portion 30 and terminates at a distance vertically below the lateral malleolus and longitudinally at the heel. Lateral support plate 28 is pivotally mounted to lateral side 34 and is of a length which at its pivoted end is slightly below the lateral malleolus and then extends to a distance above the lateral malleolus. As shown in FIGS. 3 and 6, the lateral support plate 28 terminates at the same elevation as the termination location of medial support plate 26.

FIGS. 2, 3 and 5 illustrate the preferred shapes of the medial side 32 and the lateral side 34. As illustrated each side merges upwardly from base portion 30 and tapers upwardly from a distal narrow height near the toe joints to its proximal maximum height in the heel area. The end wall of each side 32,34 extends to the end of the sole at the heel. Such end wall could be generally perpendicular to the sole 16 as indicated by the reference numbers 33 and 35 in FIGS. 2 and 5 or could be tapered, as shown in phantom by the reference numbers 33A and 35A. Each side 32, 34 in its upper region would be of a longitudinal dimension to extend longitudinally beyond its malleolus on both sides of the malleolus. Base plate 24 with its base portion 30 and upwardly sides 32, 34 is thereby generally U-shaped to receive and support a foot within its confines.

Support plates 26 and 28 are illustrated as preferably being of truncated oval shape with a rounded pivoted lower end and a straight upper end. Each support plate has an outward bulge 44 to cover and protect the respective malleolus and not impinge on the normal range of ankle motion.

Brace 14 may be made of any suitable materials which are sufficiently rigid to provide the desired support. Preferably such materials would be sufficiently light-weight so as not to unduly increase the weight of the system, as later explained. Similarly, the components of brace 14 may be of any suitable shape and dimension consistent with their intended purposes. Likewise, the various components of shoe 12 could be made of suitable size, shape and material to meet their intended functions. Most of the components could be the same as in conventional footwear.

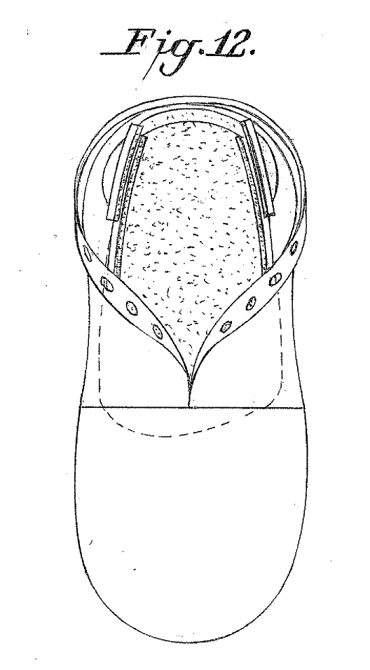

Significant features of base plate 24 are its location within the shoe 12 and its length. In that regard, as clearly shown in FIGS. 2 and 10 the base plate 24 particularly at the location of its lower base portion extends from generally the heel area to just proximal to the metatarsalphalangeal joints J of the wearer. These joints J are generally located along an arc as shown in FIG. 10. The terminal end 36 of the lower base portion 30 is also shaped to generally conform to the metatarsalphalangeal joint arc. As a result, the base plate 24 gives maximum support to the foot without interfering with toe movement. This means that the brace 14 will not restrict motion of the forefoot nor augment the strength of the joint. The base plate will wrap around the side of the foot by the provision of sides 32, 34 and a dorsal component beginning at the cuboid bone area tapering up to just proximal of the metatarsalphalangeal joint laterally. The medial aspect of the base plate will run along the side of the foot until the end of the base plate medially which is just proximal to the first metatarsalphalangeal joint of the foot.

The lateral side 34 varies from the medial side 32 in that the lateral side 34 will extend onto the dorsal area of the foot in a manner similar to a typical athletic shoe while the medial side 32 travels just along the medial side of the foot with no dorsal component.

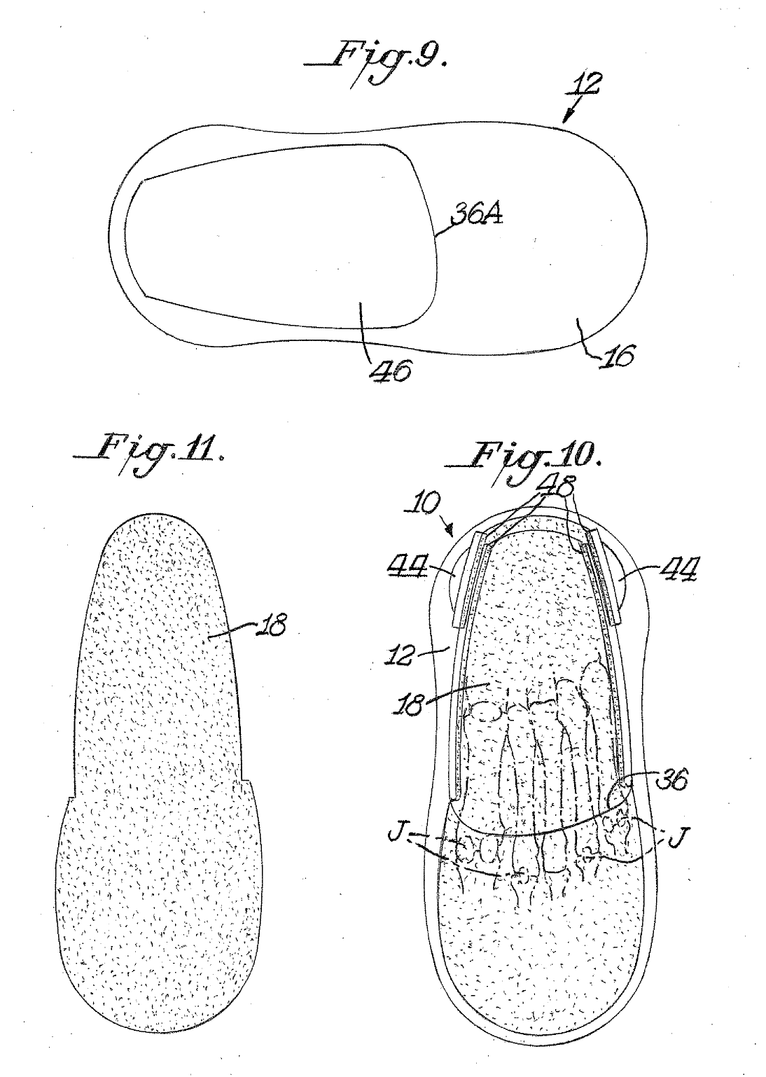

In the preferred practice of this invention each support plate 26,28 is pivotally mounted to its respective side 32, 34 by a hinge pin 38. FIG. 8 illustrates a preferred construction for the mounting of a support plate to a side. As shown in FIG. 8 the lateral side 34 has an integral hinge pin 38 connected to it. This could be done in molding step when base plate 24 is manufactured. The hinge pin 38 extends through a hole 40 in the lateral support plate 28. A cap 42 is mounted to the outer end of hinge pin 38 to maintain the lateral support plate 28 pivotally mounted to lateral side plate 34. Cap 42 may be secured in any suitable manner, such as by having a recess for snuggly receiving the exposed end of hinge pin 38. It is to be understood that other manners of securement of the cap to the hinge pin may also be done in accordance with this invention. The mounting of support plate 28 to side 34 is such as to allow sufficient clearance and thereby assure that the support plate 28 may freely rotate. Such clearance is shown by the spacing between side 34 and support plate 28 and by the spacing between support plate 28 and cap 42. A similar form of mounting would be used for pivotally securing medial support plate 26 to medial side 32.

Because the medial malleolus is lower than the lateral malleolus, FIG. 6 shows the medial hinge pin 38 to be lower than the lateral hinge pin 38. If desired, however, both hinge pins could be at the same height. Both pins, however, would be below the malleoli. In either event, as also shown in FIG. 6, both support plates 26, 28 terminate at the same height.

The hinge pins 38 which anchor the support plates to the base plate allow the shoe and foot to move in the normal flexion and extension ranges of motion. Because of the attachments to the support plates the rapid inversion forces responsible for the majority of ankle sprains is transferred away from the ankle ligaments and buffered by the force transferred to the lower leg at the malleolar level in many cases preventing or reducing the severity of the ankle sprains.

As shown in FIGS. 3, 5 and 6 the medial support plate 26 extends upwards from just below the medial hinge 38 to above the malleolar level of the shoe. In the medial support plate 26 and in the lateral support plate 28 there will be appropriate snugness of each support plate to the side of the foot and ankle while conforming to the natural bulge of the malleoli allowing normal movement to occur. Such bulge 44 in each plate is clearly shown in the various figures as completely surrounding the malleoli bulges. The shoe's padding against the malleoli allow firm but comfortable contact of the support plates to the malleoli.

The lateral support plate 28 and the medial support plate 26 are also designed with a natural contour of the range of malleolar areas so as not to restrict the foot flexion and extension motions in the shoe. As with the medial support plate 26, the lateral support plate 28 extends from just below the hinge pin 38 on the base plate 24 upwards to above the malleolus.

While the support plates 26, 28 move with the base plate 24 allowing normal flexion and extension, the support plates 26, 28 do not allow downward movement of the base plate 24 to occur independently. Rather, any downward movement is accompanied by the support plates 26, 28 resisting such movement. This will result in the most rapid transmission of support forces from the system 10 to the ankle and above the ankle when an abnormal inversion force is placed on the base plate 24.

The hinge system, which includes hinge pin 38, is preferably designed to be completely concealed within the shoe 12. If desired, however, the hinge system such as the cap 42 can be visible and/or part of the external wall of the shoe. The hinge system could be covered, for example, with a plastic covering allowing for the necessary to and fro movement of the interface of the support plates and the base plate while protecting it from external trauma.

As shown in FIGS. 2, 5 and 9 a recess 46 is formed in the sole 16. Recess 46 is of a size and shape to snuggly receive base plate 24 so that the upper surface of lower base portion 30 of base plate 24 makes a smooth transition to the upper surface of sole 16. Preferably, this combined upper surface is generally flat or planar. The insole 18 could provide any necessary arch support. By shaping the recess 46 to snuggly receive the base plate 24 there is assurance of properly mounting the brace 14 in its desired location. Recess 46 includes an arc 36A corresponding to the arc 36 at the terminal end of base plate 24. The recess 46 also has the additional benefit of reducing the weight of the shoe in the sense that material is removed from the sole. Thus, where light-weight rigid material is used for brace 14, the addition of brace 14 to the shoe 12 minimizes the weight of the combined shoe and brace. In that sense, the added weight from the brace is compensated for by the reduced shoe sole weight. Any additional weight would result from the lateral and medial support plates which can also be partially offset by using lightweight material.

In order to enhance the comfort of the ankle sprain reduction system 10 padding is preferably provided at locations where the system will contact the foot. As shown in FIGS. 2, 5 and 10, for example, the insole 18 is mounted over the upper surface of sole 16 and over the upper surface of the lower base portion 30 of base plate 24. The insole 18 (which is shown in FIG. 11) would extend the entire length of the shoe 12 at the bottom of the shoe, as with conventional insoles. Insole 18 could be loosely placed and readily removable or could be permanently mounted such as by adhesive mounting.

In addition to the padding provided by the insole 18 the system 10 also preferably includes padded or cushioning liners 48 on the inner surface of the medial and lateral side plates 32, 34 and the medial and lateral support plates 26, 28.

The ankle sprain reduction system 20 of this invention is intended to reduce the occurrence and severity of ankle sprains. This is accomplished by pivotally mounting a pair of rigid support plates to a rigid base plate. The invention takes into account, however that there may be circumstances where it is not desirable to completely resist the forces causing ankle sprains if, for example, there is too high a level of pressure, the ankle sprain reduction system 10 may be designed to operate to discontinue resisting the excessive force or pressure. This can be done by selecting as the hinge pin 38 a hinge pin made from materials and/or of dimensions so that the hinge pin would break when too much pressure or force is encountered. As a result, the provision of the system 10 would not be such that when the force is so rapid or forceful more severe damage could be incurred such as by fracturing the tibia. The hinge pin 38 could be made of any suitable diameter such as 2 or 3mm and would be designed so that when a predetermined force is exceeded the pin would fracture.

FIGS. 2 and 5 illustrate a further safety feature of system 10. As shown therein a hyperextension preventing member in the form of a stop pin or bar 50 is mounted to each side 32,34 in the general area of the heel and closer to the heel than the hinge pin 38. If the foot should pivot where the toes are bent too far, hyperextension would be prevented by the respective support plates moving into contact with and being stopped by abutting against the respective pin or bar 50.

In the preferred practice of this invention the brace 14 is manufactured as part of the shoe 12 to form the ankle sprain reduction system 10. As part of this manufacture, padding, such as from insole 18 and from cushioning liners 48, will be incorporated into the shoe. As a result, the padding will conform to the user's foot, particularly when the shoe is tightly laced. Thus, a user can select the proper size brace in the same manner that a user would ordinarily select a shoe. Because of the padding it is possible for the brace components to be completely concealed. As a result, protection is always present, particularly when the shoe is laced snuggly. Such protection is present without the structure for providing that protection to be noticeably visible. The unique design of the brace 14 within the shoe 12 is particularly efficient in the dispersion of forces especially inversion forces on the medial aspect of the foot. This is done while permitting full movement of the ankle joint and of the toes. The incorporation of the brace 14 in the shoe 12 adds minimal weight, particularly because of the recess formed in the sole to accommodate the brace and also reduce the weight of the sole.

The present invention has been described with regard to its preferred practice. It is to be understood, however, that the invention could be broadly practiced in other manners. For example, instead of incorporating the brace as a permanent part of the shoe the brace 14 could be a removable insert which would be inserted into the shoe directly above the sole. The terminal area of the brace 14 at the arc 36 could be smoothly tapered in thickness so that there is a smooth transition from the lower portion of the brace to the adjacent portion of the sole. An insole could then be placed over the sole and the lower portion of the brace. The inner surfaces of the base plate sides and of the support plates would also have padding. The invention could also be practiced where instead of making various sets of braces/shoes corresponding to the normal different incremental sizes of shoes, the braces could be provided in a fixed selection of, for example, large, medium and small sizes which are either permanently incorporated in appropriately sized shoes or which could be removably inserted into shoes dimensioned to receive such sizes.

The ankle sprain reduction system 10 thereby provides an effective system for reducing the occurrence and severity of ankle sprains wherein the system can be used by a wearer no other movements or manipulations required than be done for wearing an ordinary shoe.

1. An ankle sprain reduction system comprising a brace for being incorporated in a shoe, said brace including a rigid base plate to be mounted in the bottom of the shoe at the sole of the shoe, said base plate having a lower base portion of a length to extend from about the heel portion of the shoe to just proximal to the metatarsalphalangeal joint area of the shoe, said lower base portion having an upper surface for making a smooth transition with the upper surface of the sole adjacent to said lower base portion, a rigid medial side extending upwardly from said base portion to a distance to traveling below the medial malleolus to the heel, a rigid medial support plate pivotally mounted to and extending upwardly from said medial side, said medial support plate being pivotally mounted to said medial side by a medial hinge mechanism to be located beneath the medial malleolus with said medial support plate extending below said medial hinge mechanism and against said medial side, a rigid lateral side extending upwardly from said base portion at a location opposite from said medial side plate whereby said base plate is of generally U-shape to receive the wearer's foot between said lower base portion and said medial side and said lateral side, said lateral side extending upwardly from said lower base portion to a distance to traveling below the lateral malleolus to the heel, and a rigid lateral support plate pivotally mounted to and extending upwardly from said lateral side, said lateral support plate being pivotally mounted to said lateral side by a lateral hinge mechanism to be located beneath the lateral malleolus with said lateral support plate extending below said lateral hinge mechanism and against said lateral side, said lower base portion of said base plate spanning completely across said base plate from said medial side to said lateral side throughout the length of said base plate to completely support a foot from the heel of the foot to the metatarsalphalangeal joint, the transverse end of said lower base portion remote from the heel portion terminating in a smooth continuous arc to correspond to the metatarsalphalangeal arc, and said brace functioning to prevent/minimize inversion and eversion of the foot.

2. The system of claim 1 including said shoe, said shoe having said sole and having said joint area and having said heel portion, and said brace being mounted in said shoe.

3. The system of claim 2 wherein said brace is detachably mounted in said shoe.

4. (canceled)

5. The system of claim 2 wherein said brace is permanently mounted in said shoe, said sole including a recess extending downwardly from its upper surface, said lower base portion of said base plate being snuggly received in said recess, and an insole covering said lower base portion and said recess over a length extending beyond said lower base portion.

6. (canceled)

7. The system of claim 5 wherein said upper surface of said lower base portion is generally flat and coplanar with said upper surface of said sole.

8. The system of claim 7 including a padded insole mounted above said sole and said lower base portion.

9. The system of claim 8 including padding lining the inner surfaces of said sides and said support plates.

10. The system of claim 9 wherein each of said support plates is mounted to its respective side by a hinge pin in its respective hinge mechanism.

11. The system of claim 10 wherein said hinge pin is integral with its respective side and passes through a hole in its respective support plate.

12. The system of claim 11 including a cap mounted to the end of said hinge pin opposite said side.

13. The system of claim 10 including a stop member mounted to each of said side in the general area of said heel portion of said shoe.

14. The system of claim 10 wherein each of said support plates is outwardly bulged to accommodate the malleolus.

15. The system of claim 14 wherein said hinge pin for said lateral support plate and said lateral side is at a different height than said hinge pin for said medial support plate and said medial side to correspond to the ankle anatomy.

16. The system of claim 15 wherein said lateral support plate and said medial support plate terminate at generally the same height.

17. The system of claim 9 wherein said brace is completely concealed within said shoe.

18. The system of claim 1 wherein said shoe includes an upper portion into which a foot may be inserted and adjustable fastening structure for snuggly closing said shoe about the foot.

19. The system of claim 18 in combination with a second shoe to form a set of shoes for the left foot and the right foot, and including a plurality of said sets of shoes for different size feet and a plurality of different size braces.

20. (canceled)

21. The system of claim 1 wherein said brace is heel-less above said base plate whereby said medial side and said lateral side are separated from each other by a gap therebetween.

22. The system of claim 4 wherein said medial support plate and said lateral support plate are free of physical connection to said shoe and are free of envelopment by said shoe.

23. The system of claim 1 wherein each of said medial hinge mechanism and said lateral hinge mechanism includes a breakable pin for breaking if too much force is encountered, and said pin from said lateral hinge mechanism being at a different height than said pin from said medial hinge mechanism to correspond to the ankle anatomy.
